(19) United States
(12) Patent Application Publication
(10) Pub. No.: US 2004/0093082 A1
(43) Pub. Date: May 13, 2004

(54) Mobile-bearing artificial disc replacement

(76) Inventor: Bret A. Ferree, Cincinnati, OH (US)

Correspondence Address: John G. Posa, Gifford, Krass, Groh, Sprinkle,, Anderson & Citkowski, P.C., 280 N. Old Woodward Ave., Suite 400, Birmingham, MI, 48009-5394, US

(21) Appl. No.: 10/419,378

(22) Filed: Apr. 21, 2003

Related U.S. Application Data
(60) Provisional application No. 60/373,882, filed on Apr. 19, 2002.
(60) Provisional application No. 60/438,408, filed on Jan. 7, 2003.
(60) Provisional application No. 60/443,324, filed on Jan. 29, 2003.

(51) Int. Cl. A61F 2/44

(52) U.S. Cl. 623/17.11; 606/61

(57) ABSTRACT

Artificial disc replacements (ADRs) use mobile spacers between vertebral endplates. In the preferred embodiment, spherical spacers are used between metal or ceramic plates placed over or in the endplates of the vertebrae. The spherical spacers may be made of metal, ceramics or polymers such as polyethylene. One or more of the bearings may be used in each disc replacement, and one or more disc replacements may be inserted into the disc space. The small bearings allow the preservation of the vertebral endplates. Although the preferred embodiment use mobile units preferably in the form of small spheres and two ADRs per level, alternatives are disclosed, wherein the mobile unit is non-spherical, including oblong shapes. Further alternative configurations include an elongated mobile bearing contained by interdigitating projections from superior and/or inferior endplates.

### REFERENCE TO RELATED APPLICATION

This application claims priority from U.S. Provisional Patent Application Serial Nos. 60/373,882, filed Apr. 19, 2002; 60/438,408, filed Jan. 7, 2003; and 60/443,324, filed Jan. 29, 2003. The entire content of each application is incorporated herein by reference.

### FIELD OF THE INVENTION

This invention relates generally to artificial disc replacements (ADRs) and, more particularly, to mobile-bearing ADRs.

### BACKGROUND OF THE INVENTION

Mobile spacers between joint surfaces have important advantages over non-mobile spacers. First, they increase the allowed motion. Second, mobile bearings decrease surface wear.

Various mobile spacers have been applied to total-knee replacements. Prior-art mobile bearing total joint replacement prostheses are described in Noiles, U.S. Pat. No. 4,219,893, Goodfellow and O'Connor, U.S. Pat. No. 4,085,466, and Buechel and Pappas U.S. Pat. Nos. 4,309,778 and 4,340,978. Noiles, in particular describes a mobile bearing knee in which a bearing is retained by side walls of a tibial platform. The inner face of the side walls of the Noiles tibial platform are circular cylinders, as is the complementary side wall of the bearing.

Artificial disc replacements (ADRs) with moving components have also been tried. According to U.S. Pat. No. 4,759,766, an intervertebral disc endoprothesis comprises two symmetrical, concave endplates with an intermediate convex spacing piece. The endplates and the spacing piece have a plane guide rim. The end plates either have an edge shoulder or an annular groove for a toroid provided on the spacing piece. Alternatively, the intervertebral disc endoprothesis comprises two asymmetric end plates and a spacing piece. In the third variation, the two symmetrical end plates are convex, the spacing piece is cylindrical as well as concave at the two ends and has a durable cover.

Improvements have been made over the years, but the resultant devices teach the use of mobile spacers that articulate with endplates by way of congruent surface. One example, disclosed in U.S. Pat. No. 6,368,350, is directed to intervertebral prosthetic devices and methods providing a variable instantaneous axis of rotation. In general, the disclosed devices include two bearing surfaces, a first bearing surface being curved and a second bearing surface being planar. In some embodiments, the curved bearing surface provides at least three degrees of rotational freedom and the planar bearing surface provides at least two degrees of translational freedom and one degree of rotational freedom. Several embodiments with varying degrees of rotational or translational freedom are disclosed.

### SUMMARY OF THE INVENTION

The present invention uses mobile spacers between vertebral endplates. In the preferred embodiment, spherical spacers are used between metal or ceramic plates placed over or in the endplates of the vertebrae. The spherical spacers may be made of metal, ceramics or polymers such as polyethylene. One or more of the bearings may be used in each disc replacement, and one or more disc replacements may be inserted into the disc space. The small bearings allow the preservation of the vertebral endplates.

Although the preferred embodiment use mobile units preferably in the form of small spheres and two ADRs per level, alternatives are disclosed, wherein the mobile unit is non-spherical, including oblong shapes. Further alternative configurations include an elongated mobile bearing contained by interdigitating projections from superior and/or inferior endplates.

### BRIEF DESCRIPTION OF THE DRAWINGS

FIG. 1A is a side-view drawing of a first embodiment of the invention;

FIG. 1B is an end-view drawing of the structure of FIG. 1A;

FIG. 2A is a drawing of an embodiment of the invention utilizing three laterally movable elements in a neutral condition;

FIG. 2B is a lateral view of the embodiment of FIG. 2A;

FIG. 2C is a lateral view showing the way in which the mobile units move to accommodate extension;

FIG. 3A is a side-view drawing of an alternative embodiment of the invention in cross-section;

FIG. 3B is an end-view drawing of the structure of FIG. 3A;

FIG. 3C is an anterior view of this alternative embodiment illustrating extension;

FIG. 3D is a lateral view of this alternative embodiment showing flexion;

FIG. 4A is an anterior view of a different embodiment of the invention;

FIG. 4B is a lateral view of the embodiment of FIG. 4A;

FIG. 4C is an anterior cross-section of the structures of FIGS. 4A and 4B;

FIG. 4D is a lateral cross-section of the structure of FIGS. 4A-4C;

FIG. 5A is a drawing which shows the structure of FIG. 4 in extension;

FIG. 5B shows the structure of FIG. 4 in flexion;

FIG. 6 is an axial cross-section of the device of FIGS. 4 and 5;

FIG. 7A shows how the end plates may be wedge-shaped or trapezoidal to provide for lordosis;

FIG. 7B is a more detailed drawing of the device of FIG. 7A;

FIG. 8A is a drawing which shows an endplate according to the invention using spikes;

FIG. 8B is a drawing which shows an endplate according to the invention held in position using a diagonally oriented screw;

FIG. 8C is a drawing which shows an endplate according to the invention;

FIG. 9A shows a first step associated with implanting an ADR according to the invention;

FIG. 9B illustrates a step of inserting a second opposing end plate;

FIG. 9C illustrates how the mobile units may then be installed;

FIG. 9D illustrates the step of securing end caps through the end plates;

FIG. 9E illustrates the assembled mobile bearing apparatus in place;

FIG. 10A is a side-view drawing of yet a further alternative mobile bearing artificial disc replacement insert according to the invention;

FIG. 10B is an end-view of the embodiment of FIG. 10A;

FIG. 11A is shows a modification of the device of FIG. 10;

FIG. 11B is an end view of the device showing both of these optional modifications;

FIG. 12A is a side-view drawing of the device of FIGS. 10 or 11 in place;

FIG. 12B is an end view drawing showing two of the devices of FIGS. 10 or 11 in places;

FIG. 13A is a drawing which illustrates a tool used to screw in certain of the ADR embodiments disclosed herein;

FIG. 13B is an end view of the tool of FIG. 13A;

FIG. 14A is an anterior aspect of an alternative embodiment of an ADR according to the invention;

FIG. 14B is a view of the lateral aspect of the embodiment of the ADR drawn in FIG. 14A;

FIG. 14C is a coronal cross section of the embodiment of the ADR drawn in FIG. 14A;

FIG. 14D is a sagittal cross section of the embodiment of the ADR drawn in FIG. 14A;

FIG. 14E is a sagittal cross section of the embodiment of the ADR drawn in FIG. 14D;

FIG. 15A is a sagittal cross section of an alternative embodiment of the ADR drawn in FIG. 14;

FIG. 15B is a coronal cross section of the ADR drawn in FIG. 15A;

FIG. 15C is a coronal cross section of the ADR drawn in FIG. 5A and a flexed spine;

FIG. 16A is a sagittal cross section of a mechanism for containing mobile bearings inserted in situ into ADRs according to this invention;

FIG. 16B is an exploded sagittal cross section of the ADR drawn in FIG. 16A. FIG. 16C is an axial cross section of the top ADR EP drawn in FIG. 16A;

FIG. 17 is an axial cross section of the spacer of FIG. 16A and a tool used to hold the spacer;

FIG. 18A axial cross section with sliding components shown in a position that facilitates insertion of the spacer component;

FIG. 18B is an axial cross section of the ADR drawn in FIG. 18A;

FIG. 19A coronal cross section with a spacer component shown during insertion between the ADR EPs; and

FIG. 19B is a coronal cross section of the ADR drawn in FIG. 19A.

### DETAILED DESCRIPTION OF THE INVENTION

FIG. 1A is a side-view drawing of a first embodiment of the invention, wherein a plurality of thoroughly movable elements, preferably ball bearings 106, are disposed between end plates 102, and contained within a cavity created between the end plates using check reins 104. Preferably, the superior and inferior surfaces of the end plates 102 include some form of roughening, projections, or other features to assist in maintaining the structure between the upper and lower vertebrae. FIG. 1B is an end-view drawing of the structure of FIG. 1A. Although two side-to-side units are shown, more or fewer may be used, and may be used in conjunction with non-mobile-bearing configurations, depending upon the particular application.

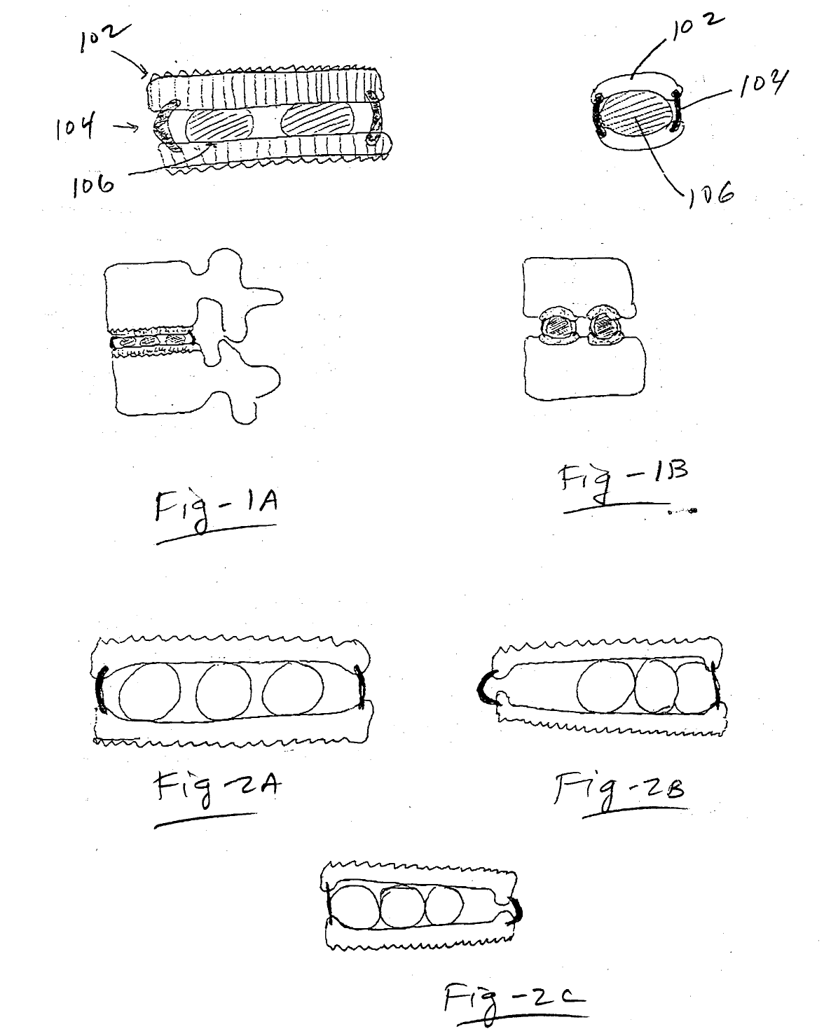

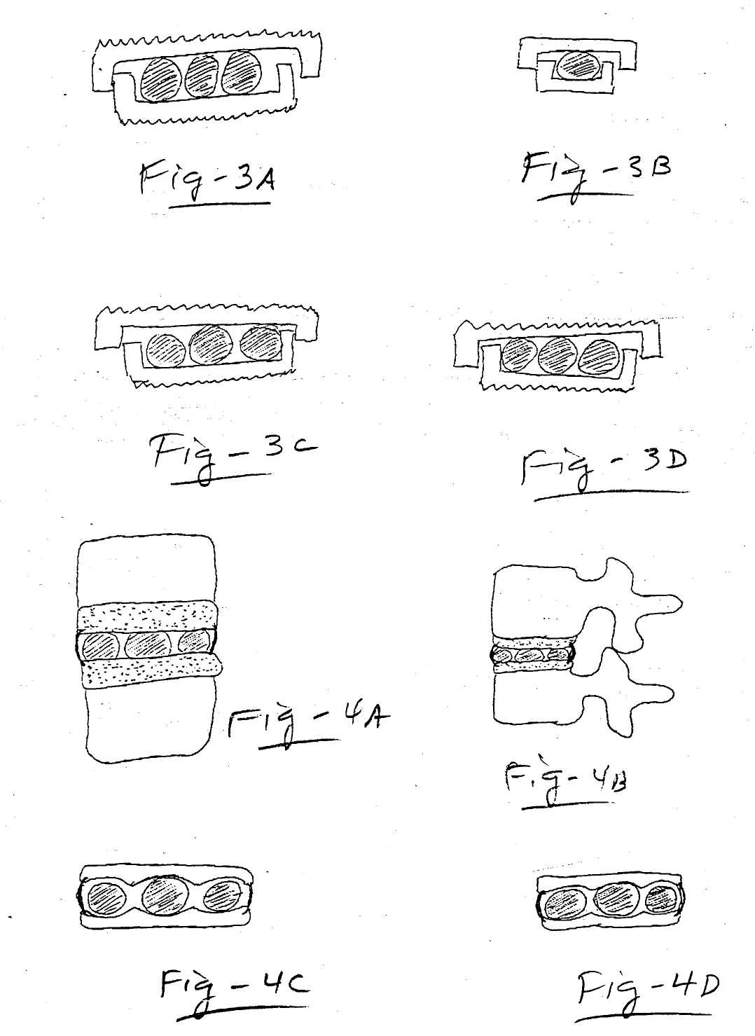

FIG. 2A is a drawing of an embodiment of the invention utilizing three laterally movable elements in a neutral condition. FIG. 2B is a lateral view of the embodiment of FIG. 2A, illustrating the way in which the mobile units, in this case, spheroids, move to accommodate flexion. Note also how the check reins expand and impress to accommodate such movement. FIG. 2C is a lateral which shows the way in which the mobile units move to accommodate extension.

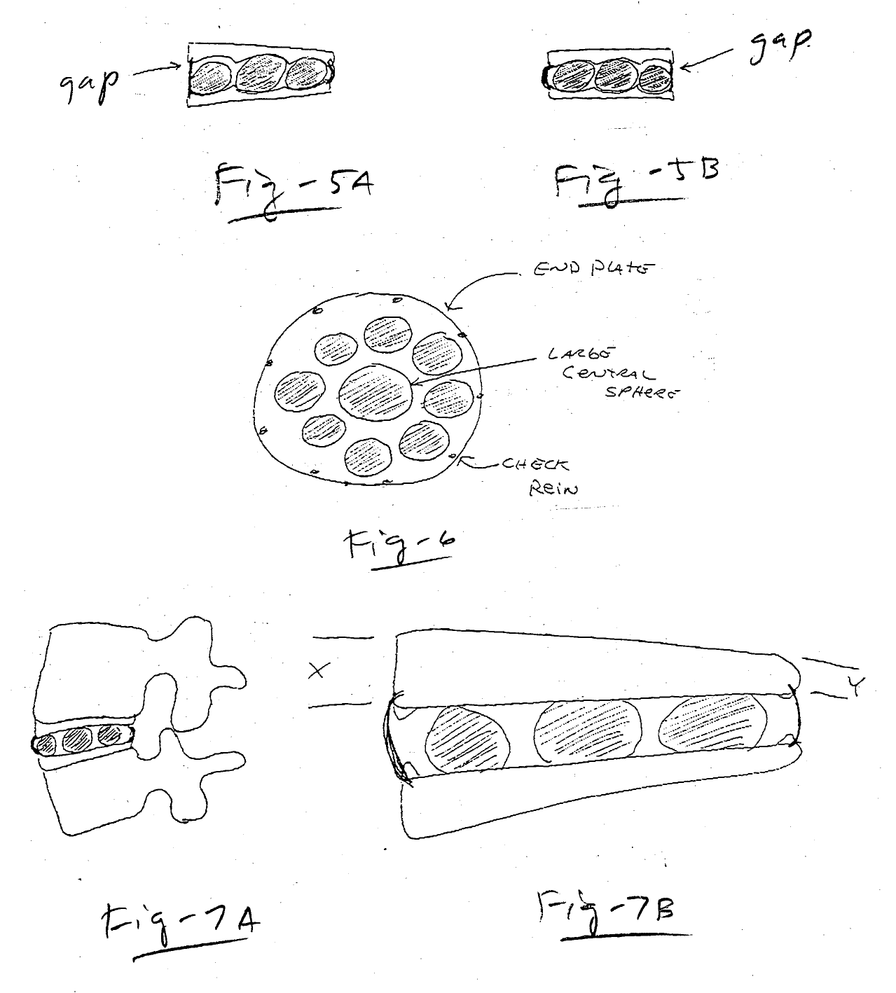

FIG. 3A is a side-view drawing of an alternative embodiment of the invention in cross-section, showing how overlapping end plates may be used as a substitute for check reins. FIG. 3B is an end-view drawing of the structure of FIG. 3A. While the embodiment of FIGS. 3A and 3B illustrate mobile units in the form of substantially equal sides spheroids, FIG. 3C illustrates a further embodiment of the invention, wherein one or more of the central spheroids are larger in diameter than the outer units to facilitate extension and flexion. FIG. 3C is an anterior view of this alternative embodiment illustrating extension, and FIG. 3D is a lateral view of this alternative embodiment showing flexion.

FIG. 4A is an anterior view of a different embodiment of the invention, wherein the movement of the mobile units is at least partially constrained through upper and lower cavitations. FIG. 4B is a lateral view of the embodiment of FIG. 4A. FIG. 4C is an anterior cross-section of the structures of FIGS. 4A and 4B, and FIG. 4D is a lateral cross-section of the structure of FIGS. 4A-4C. Note that, again, this particular embodiment of the central mobile bearing member, in this case a spheroid, is made larger to allow for flexion, extension and lateral bending.

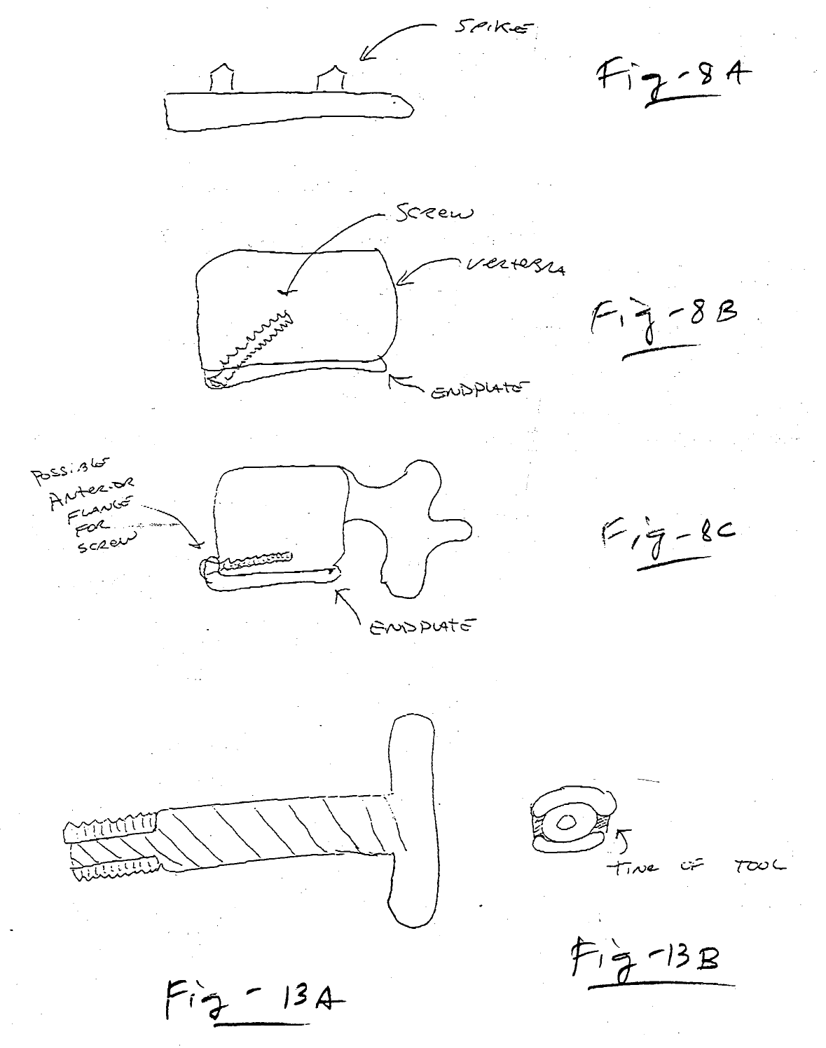

FIG. 5A is a drawing which shows the structure of FIG. 4 in extension, and FIG. 5B shows the structure of FIG. 4 in flexion. Note that, particularly through the use of a larger central mobile unit, an anterior gap occurs in extension, whereas a posterior gap occurs in flexion. FIG. 6 is an axial cross-section of the device of FIGS. 4 and 5, showing the way in which the mobile elements may be arranged relative to the end plates. Note that although a single larger central sphere is shown surrounded by smaller spheres in a more or less circular concentric arrangement, the end plates and geometry of the mobile unit positioning need not be symmetrical, but may be distributed for a more anatomical relationship in accordance with position along the spine and other factors. The check reins are shown at 602.

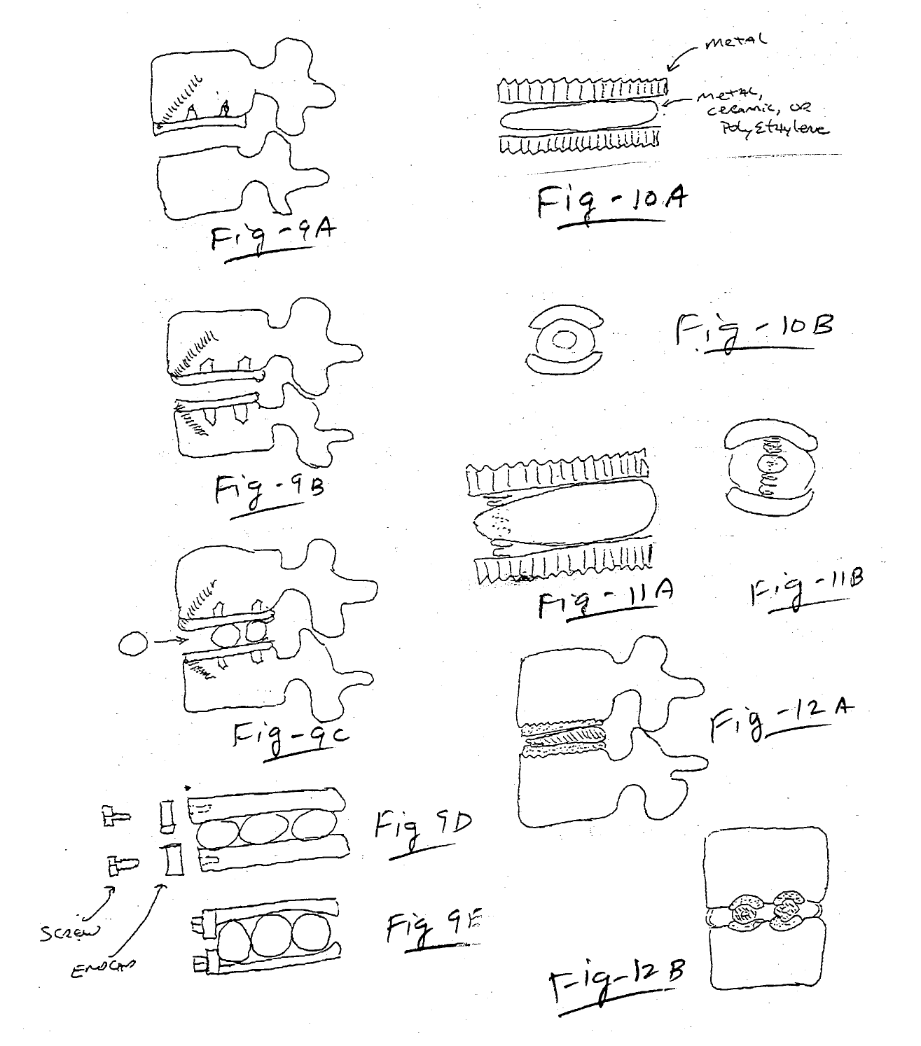

Additionally, although the end plates may be of uniform or consistent lateral thickness, FIG. 7A shows how the end plates may be wedge-shaped or trapezoidal to provide for lordosis. FIG. 7B is a more detailed drawing of the device of FIG. 7A, showing how one edge X is made larger than an opposing edge Y to account for this geometry.

In addition to the use spikes, protrusions, surface roughening, and the like, the end plates may be covered partially or entirely with a bone-ingrowth surface to enhance fixation. In addition, the end plates may have larger projections into the vertebrae, such as spikes or screws. FIG. 8A is a drawing which shows an endplate according to the invention using spikes. FIG. 8B is a drawing which shows an endplate according to the invention held in position using a diagonally oriented screw. FIG. 8C is a drawing which shows an endplate according to the invention which shows the use of an optional anterior flange to facilitate a different form of fixation.

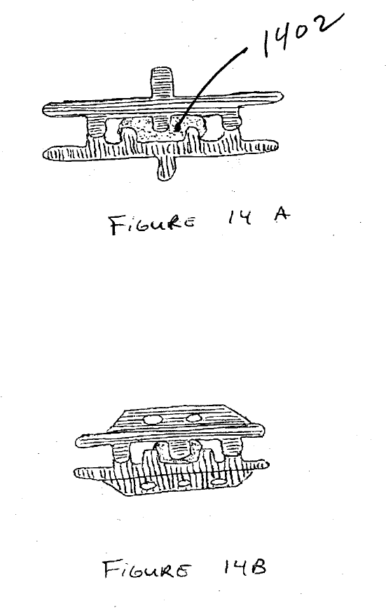

FIGS. 9A-9E illustrate a preferred way in which a mobile-bearing artificial disc replacement (ADR) system may be assembled into a disc space. FIG. 9A shows a first step associated with implanting an ADR according to the invention, wherein a first end plate is inserted. FIG. 9B illustrates a step of inserting a second opposing end plate, both being screwed into place. FIG. 9C illustrates how the mobile units may then be installed, assuming some form of distraction is used to keep the end plates sufficiently apart. FIG. 9D illustrates the step of securing end caps through the end plates to ensure that the mobile units remain in place. FIG. 9E illustrates the assembled mobile bearing apparatus in place.

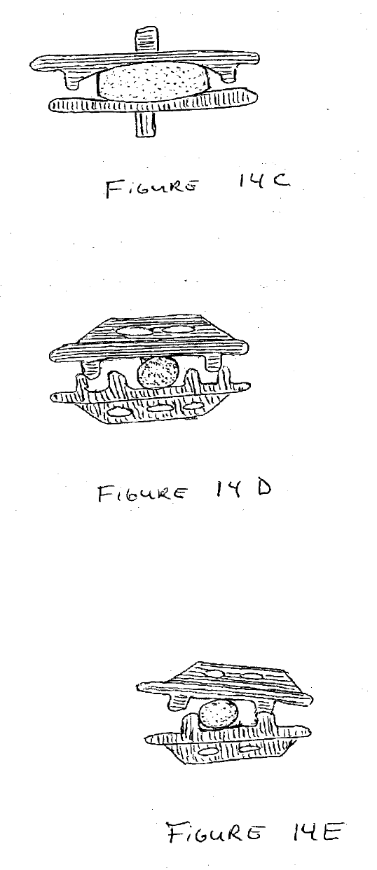

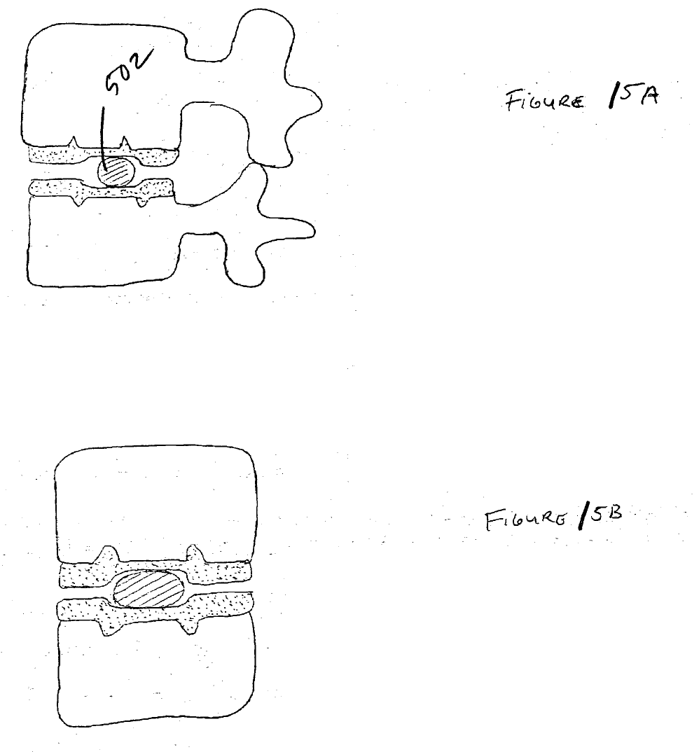

FIG. 10A is a side-view drawing of yet a further alternative mobile bearing artificial disc replacement insert according to the invention, wherein the mobile unit is oblong as opposed to spherical. FIG. 10B is an end-view of the embodiment of FIG. 10A. FIG. 11A is shows a modification of the device of FIG. 10, including the use of an anterior spring in conjunction with a posterior enlargement to prevent or limit extension. FIG. 11B is an end view of the device showing both of these optional modifications. FIG. 12A is a side-view drawing of the device of FIGS. 10 or 11 in place. FIG. 12B is an end view drawing showing two of the devices of FIGS. 10 or 11 in places, with the understanding that more or fewer may be used as discussed above. FIG. 13A is a drawing which illustrates a tool used to screw in certain of the ADR embodiments disclosed herein. FIG. 13B is an end view of the tool of FIG. 13A. As seen in the figures, the tool slips over the ADR to allow for easy removal of the tool once the ADR is placed.

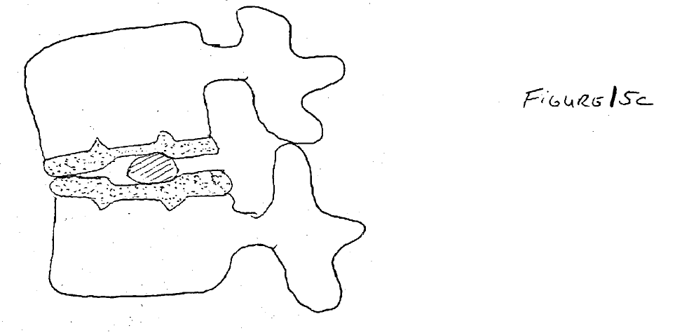

FIG. 14A is a view of the anterior aspect of an alternative embodiment of the invention, wherein an elongated mobile bearing 1402 is contained by interdigitating projections from the superior and/or inferior ADR EPs. In this embodiment the projections do not restrict normal spinal motion. Alternatively, the projections could interact to limit spinal motion. For example, the projections could interact to limit excessive spinal rotation.

FIG. 14B is a view of the lateral aspect of the embodiment of the ADR drawn in FIG. 14A. Note that the elongated mobile bearing may have a smaller radius when viewed from the side (FIG. 14B) than when the bearing if viewed from the front (FIG. 14A).

FIG. 14C is a coronal cross section of the embodiment of the ADR drawn in FIG. 14A better illustrating the line of contact between the mobile bearing and the ADR EPs. FIG. 14D is a sagittal cross section of the embodiment of the ADR drawn in FIG. 14A. FIG. 14E is a sagittal cross section of the embodiment of the ADR drawn in FIG. 14D. The drawing illustrates movement of the bearing during movement of the ADR. The drawing also illustrates retention of the bearing by the projections from the ADR EPs.

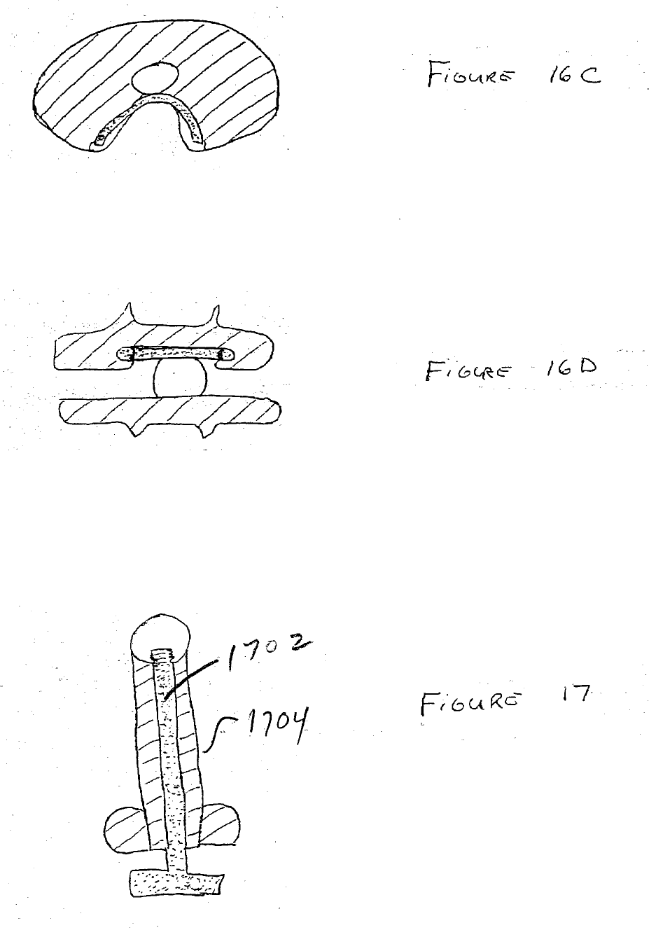

FIG. 15A is a sagittal cross section of an alternative embodiment of the ADR drawn in FIG. 14. A mobile component 502 cooperates with ADR endplates and allows tilting and sliding to achieve spinal motion. FIG. 15B is a coronal cross section of the ADR drawn in FIG. 15A and the spine. FIG. 15C is a coronal cross section of the ADR drawn in FIG. 15A and a flexed spine. The mobile component may migrate posteriorly during spinal flexion. Posterior migration of the mobile component eases spinal flexion and reduces distraction of the posterior portion of the ADR during spinal flexion. The ADR endplates can impinge to limit motion.

FIGS. 16 through 19, taken from U.S. Provisional Patent Application Serial No. 60/443,324 illustrate alternative mechanisms for containing mobile bearings inserted in situ into ADRs according to this invention. FIG. 16A is a sagittal cross section of one such mechanism, wherein a removable clip component 1602 holds a removable spacer component 1604 in position between the ADR EPs. FIG. 16B is an exploded sagittal cross section of the ADR drawn in FIG. 16A. FIG. 16C is an axial cross section of the top ADR EP drawn in FIG. 16A. The removable clip fits into a slot in the ADR EP. FIG. 16D is a coronal cross section of the ADR drawn in FIG. 16A.

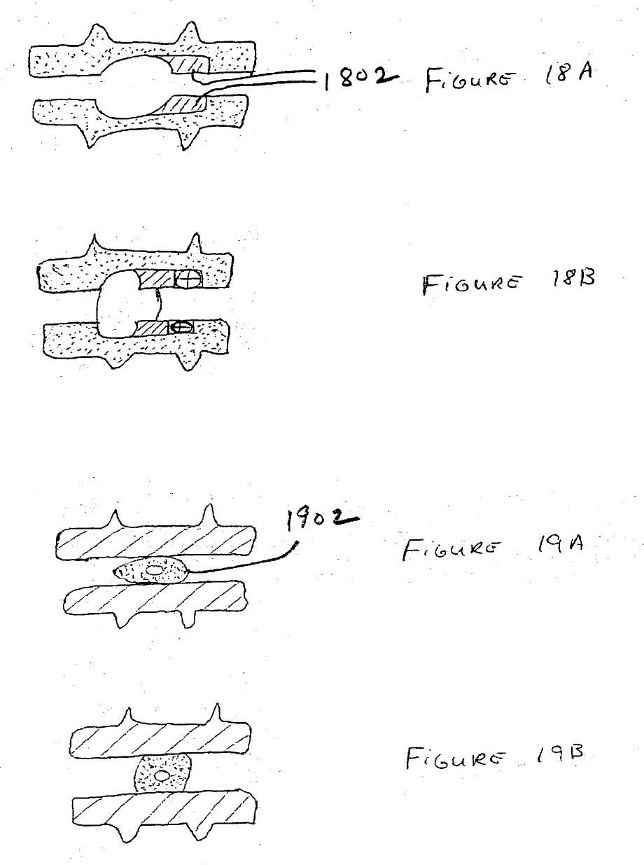

FIG. 17 is an axial cross section of the spacer of FIG. 16A and a tool used to hold the spacer. A component 1702 of the tool is threaded into the spacer component. A second component 1704 of the tool is fitted over the spacer to prevent rotation of the spacer while inserting and removing the threaded component of the tool.

In the axial cross section of FIG. 18A, sliding components 1802 are shown in a position that facilitates insertion of the spacer component. FIG. 18B is an axial cross section of the ADR drawn in FIG. 18A, with the sliding components in a position that blocks extrusion of the spacer component. The sliding components can be held in the closed position with screws that are threaded into the ADR EPs. The screw threads can deform to prevent screw loosening.

In the coronal cross section of FIG. 19A, the spacer component 1902 is shown during insertion between the ADR EPs. The spacer component is inserted with its long axis parallel to the opening in the ADR EPs. The hole in the center of the spacer component can be used by an insertion tool. The hole within the spacer component may also allow the spacer component to reversibly deform with spinal movement. FIG. 19B is a coronal cross section of the ADR drawn in FIG. 19A. The spacer component is shown in its final position. Rotation of the spacer component 90 degrees from the insertion position to the final position cams the ADR EPs apart to distract the vertebrae.

Summarizing, embodiments of this invention are directed to mobile-bearing ADRs wherein the articulating surfaces between the mobile bearing and the ADR endplates (EP) are not congruent. In contrast to existing devices, this permits the mobile bearing to articulate and slide relative to the ADR EPs, which in turn allows the mobile bearing to “self-center” during spinal movement.

One or more mobile bearings may be used per ADR according to the invention, and the range of movement of the mobile bearing can extend over most of the ADR EP surface, or be limited to a portion of the ADR EPs. For example, the movement of the mobile bearing may be limited to the posterior half of the ADR EPs. The mobile bearing may be restricted to less than 1 mm of movement or permitted to move 3-5 cm or more.

As disclosed, the mobile bearing may be a sphere or elongated body with two or more radii. Alternatively, the mobile bearing could be a cylinder. An embodiment of the ADR with a spherical mobile bearing has point contact between the mobile bearing and the ADR EPs, whereas the embodiment with an elongated mobile bearing has line contact between the mobile bearing and the ADR EP.

Overall, it is believed that ADRs according to the invention reproduce the kinematics of the natural disc better than most prior art ADRs by facilitating all the normal spinal movements including translation. Also disclosed is the use of check reins to a) permit movement of the ADR, and b) retain the mobile bearings.

The ADR can be inserted fully assembled (as in FIG. 14) or assembled in-situ (FIG. 9), as disclosed in pending U.S. Provisional Patent Application Serial No. 60/438,408, incorporated herein by reference in its entirety. This is one of the reasons why, in contrast to single-component ADRs utilizing endplates constructed of a single material, assembled ADRs according to this invention allow the use of more than one material. Thus, materials with good wear characteristics can be combined with materials exhibiting other desirable characteristics such as the elasticity, shape-memory, and so forth. Devices according to the invention may also be used for other joints of the body, such as prosthetic knees and hips.

I claim:

1. A mobile bearing artificial disc replacement (ADR) for use in the disc space between the inferior endplate of an upper vertebral body and the superior endplate of a lower vertebral body, the ADR comprising:
a first plate having an upper surface attached to the inferior endplate of the upper vertebral body and a lower surface facing into the disc space;
a second plate having a lower surface attached to the superior endplate of the lower vertebral body and an upper surface facing into the disc space; and
one or more spacers laterally moveable between the surfaces of the plates facing into the disc space.

2. The mobile bearing ADR according to claim 1, wherein one or more of the mobile units are spherical.

3. The mobile bearing ADR according to claim 1, wherein one or more of the mobile units are cylindrical or oblong.

4. The mobile bearing ADR according to claim 1, wherein one or both of the plates are wedged-shaped to account for lordosis or other anatomical features.

5. The mobile bearing ADR according to claim 1, further including one or more flexible guards spanning the opposing plates to retain the spacers therebetween.

6. The mobile bearing ADR according to claim 1, wherein the first and second plates interdigitate.
